(19) United States
(12) Patent Application Publication
(10) Pub. No.: US 2016/0045657 A1
(43) Pub. Date: Feb. 18, 2016

(54) METHOD OF ADJUSTING BLOOD FLOW IN A DIALYSIS MACHINE AND DIALYSIS MACHINE

(71) Applicant: B. BRAUN AVITUM AG, MELSUNGEN (DE)

(72) Inventors: SILVIE KRAUSE, MELSUNGEN (DE); CHRISTOF STROHHOEFER, KASSEL (DE)

(21) Appl. No.: 14/821,259

(22) Filed: Aug. 7, 2015

(30) Foreign Application Priority Data
Aug. 14, 2014 (DE) 10 2014 111 665.8

(51) Int. Cl. A61M 1/36

(52) U.S. Cl. CPC A61M 1/3639

(57) ABSTRACT

Methods for the adjustment of a blood flow in a blood treatment machine/dialysis machine are disclosed. Steps for achieving a blood flow having an optimum value include determining a blood flow target value, altering the blood flow at a predetermined blood flow alteration rate, comparing a venous pressure with a venous pressure threshold, an arterial pressure with an arterial pressure threshold, and the blood flow with the blood flow target value, determining if a dialysis fluid parameter extreme is reached, storing an optimum blood flow value, in dependence on a blood flow (according to a value which is stored in a data table and which takes into account the blood flow target value as well as the measurement lag) in an optimum blood flow value memory, for which the dialysis fluid parameter threshold, the venous pressure threshold or the arterial pressure threshold, or the blood flow target value has been reached.

### CROSS REFERENCE TO RELATED APPLICATIONS

This application claims priority to German application DE 10 2014 111 665.8 filed Aug. 14, 2014, the contents of such application being incorporated by reference herein.

### FIELD OF THE INVENTION

The present invention relates to the adjustment of a blood flow in an extracorporeal blood treatment machine, preferably a dialysis machine, and in particular to a (machine control) method for adjusting the blood flow as well as to an extracorporeal blood treatment/cleansing machine, preferably a dialysis machine with a control device or control unit for adjusting the blood flow making use of the (control) method according to aspects of the present invention.

### BACKGROUND OF THE INVENTION

For extracorporeal blood treatment/blood cleansing, such as dialysis, blood flow (Qb) adjustment is of great importance to the efficiency of the treatment. For example, dialysis patients have an artificial puncture site or an artificial access to the intracorporeal blood vessel system, which may consist either of a shunt (a connection between vein and artery) or a central venous catheter. At this puncture site/vascular access, blood for the dialysis treatment is removed from the patient's body. Blood flow is normally set as high as possible, since a higher blood flow is generally associated with a higher cleansing performance and results thus in a better detoxification performance for the therapy and/or in a reduction of the duration of treatment.

During a dialysis treatment, complications or certain undesired phenomena may occur. Some of them have the effect that the assumption that a higher blood flow will lead to a higher cleansing performance is no longer correct. An example for this is the occurrence of a so-called recirculation or local shunt recirculation. The recirculation or shunt recirculation (R) is defined as the ratio of the flows of recirculated blood (Qr) and the blood pump rate or total blood flow (Qb). Hence, what is referred to as recirculated blood is the blood which has already been cleansed and which comes from the venous needle (blood return line), said blood, together with patient blood that has not yet been cleansed, being conveyed (recirculated) directly back into the arterial needle (blood supply line). The recirculation or shunt recirculation (R) is indicated in percent:

R=Qr/Qb.

The medically induced blood flow depends, inter alia, on the condition of the access to the patient and the shunt, respectively. Hence, the physician in charge finds himself confronted with the task of adjusting the blood flow in extracorporeal blood treatment as high as possible, on the one hand, so as to achieve the highest possible cleansing performance for a therapy, and to avoid, on the other hand, an excessively high blood flow so as not to risk recirculation or so as to keep recirculation as small as possible.

### DESCRIPTION OF THE RELATED ART

The prior art discloses the following methods and machines for extracorporeal blood treatment/cleansing, such as dialysis.

WO 2007/140993 describes a device for controlling an extracorporeal blood treatment machine. This blood treatment machine makes use of at least one predetermined flow rate from a group of flow rates comprising the blood flow rate Qb, the dialysis fluid rate Qd, the ultrafiltration rate Qf and the substituent rate Qs, for calculating, only on the basis of a predetermined dependence of the clearance K or the dialysance D on flow rates, at least one of the respective other flow rates from the group of flow rates comprising the blood flow rate Qb, the dialysis fluid rate Qd, the ultrafiltration rate Qf and the substituent rate Qs, at which the predetermined clearance K or dialysance D is maintained.

U.S. Pat. No. 3,882,861 describes a dialysis machine with the aid of which the blood flow is adapted in a pressure-controlled manner. The blood flow is controlled by a sequence of electrical pulses, in the case of which the respective pulse duration corresponds to changes in the negative pressure occurring when there is a change in the blood flow. This solution is, however, disadvantageous insofar as the suggested machine is technically complicated and therefore expensive. Moreover, the applicant of the present invention noticed that such a machine, which operates exclusively in a pressure-controlled manner, cannot necessarily guarantee that the best possible cleansing performance of the treatment will be accomplished.

EP 0 711 182 B1 describes a system for accomplishing the highest possible clearance value with respect to the patient's whole body. The system comprises a unit for adjusting a dialysis efficiency parameter, a unit for detecting a metabolite concentration, a unit for ascertaining a metabolite profile in dependence on the varied parameter, and a unit for comparing the measured metabolite concentration values, so as to determine an optimum parameter with which a maximum metabolite concentration can be accomplished.

One disadvantage of this prior art is to be seen in that the determination of the urea concentration in the outgoing fluid is utilized for obtaining information with respect to the blood flow at which the dialysis-fluid-side toxin concentration (here urea concentration) is at its maximum. This measurement necessitates an adjustment of various blood flows. After a change in blood flow, the measurement cannot be carried out on the dialysis fluid side until a stable value has been established, i.e. after the end of the compensation process, within the extracorporeal blood line system of the machine. Since the patient's dialysis is, however, continued without interruption during the measurements, it is doubtful that the correct blood flow can actually be ascertained, since the measurement times would have to be very high/long. The device and the suggested method are therefore not necessarily suitable for quickly ascertaining the blood flow at which a (substantially) maximum removal of toxins is possible.

EP 1 083 948 B1 describes a method for determining, on the basis of transmission spectroscopy, the concentration of waste products, i.e. filterable uremic toxins, in the dialysis fluid during a dialysis treatment. The measurement is carried out by means of a spectrophotometer and the measurement result is multiplied by the through-flow from the dialyzer so as to determine the content of the substance(s) in the outgoing dialysis fluid.

This allows measurement of the absorbance of a mixture of substances existing on the dialysis fluid side. The data are, however, not used for drawing any conclusions with respect to the blood flow.

Finally, WO 2013/167264 describes a method and a device for extracorporeal blood treatment, which is intended to be used for accomplishing an optimization of a blood flow rate to be preset, in the sense of a maximization of the exchange performance of a dialyzer. To this end, the device as well as the method according to this prior art provide the determination of at least one, preferably of a plurality of parameters that are characteristic of an extracorporeal blood treatment, a specific blood flow rate being determined in each case in dependence on the one, or preferably of one of the plurality of characteristic parameters.

Subsequently, a blood flow rate is selected from a plurality of blood flow rates that have been determined on the basis of the characteristic parameters, said blood flow rate being then preset for the current treatment. The selection of said one blood flow rate is executed making use of a (selection) algorithm implemented in a device-internal software/hardware. The algorithm allows automatic selection of said one blood flow rate.

### SUMMARY OF THE INVENTION

Taking into account this known prior art, it is an object of the present invention to provide a (machine control) method for adjusting/achieving a blood flow for a substantially maximum cleansing performance, and to create an extracorporeal blood treatment machine/cleansing machine, preferably a dialysis machine, which is adapted to be used for adjusting an optimum blood flow (for a substantially maximum cleansing performance) in a dialysis treatment. One object is to allow the blood flow to be adjusted such that recirculation, e.g. in a patient's shunt, will be reduced or avoided. Another object is to configure the method and the device, in which the method is implemented, as simple as possible.

This object is achieved by the (machine control) claimed method for adjusting a blood flow and the claimed extracorporeal blood treatment/cleansing machine (dialysis machine). Preferred embodiments of the present invention are the subject matter of the respective subclaims.

Summarizing, it can be stated that the invention relates to the general process, according to which the blood flow through the dialyzer is increased, preferably linearly, at a predetermined rate (i.e. within a specific (process) time t starting from a predetermined initial value to a predetermined target value), the current venous and/or arterial pressure in the extracorporeal blood circuit as well as dialysis-side features/characteristics (in particular the current degree or amount of uremic toxins) in a spent cleansing fluid (dialysis fluid) being measured, continuously or in a clocked mode, with suitable sensors. These concrete measurement values can then be used for determining/ascertaining, preferably by a comparison between the detected measurement values and (standardized) target values which have been adjusted in advance or which have already been implemented, the (individual) blood flow that is most advantageous for the treatment carried out at the time in question.

The detection, especially the detection of the above-mentioned, dialysis-side features/characteristics entails a dead time Δt (the actually occurring delay time between the rate alteration made and the result of such alterations measurable at the sensors), which results substantially from the distance between the dialyzer and the sensor in the longitudinal direction of the tube as well as from the (average) flow velocity of the cleansing fluid. This dead time Δt must be incorporated and taken into consideration in the determination routine, so as to take the final decision with respect to the optimum blood flow adjustment.

The (machine control) method according to aspects of the present invention used for adjusting a blood flow for a substantially optimum cleansing performance in an extracorporeal blood treatment/cleansing machine, preferably a dialysis machine, comprises, expressed more concretely, the following steps:
a) predetermining a blood flow target value, Qb_target, preferably via a communication unit,
b) altering a (predetermined/predeterminable) initial blood flow, Qb_start (different from Qb_target), at a predetermined/predeterminable blood flow alteration rate and thus over a predetermined maximum blood flow alteration period t in the direction of the blood flow target value (Qb_target), e.g. through a blood pump/control unit,
c) comparing a measured current venous pressure PV with a (predetermined or selected) venous pressure threshold; a measured current arterial pressure PA with a (predetermined or selected) arterial pressure threshold; and a measured current blood flow Qb with the blood flow target value, Qb_target, with a comparator unit,
d) detecting at least one current dialysis fluid parameter/feature/characteristic (degree/amount of uremic toxins contained in a spent dialysis fluid, e.g. via UV-absorption/absorbance) with a time delay/dead time Δt with respect to the moment in time of the associated measured blood flow (in dependence on the flow velocity of the dialysis fluid as well as on the flow distance between the detection site and the dialyzer) through a detection unit and determining through a determination unit whether the detected current dialysis fluid parameter approaches/reaches a dialysis fluid parameter threshold (determination of the occurrence of a parameter extreme),
e) storing an optimum blood flow value, Qb_optimum, in an optimum blood flow value memory, in dependence on the blood flow or the blood flow rate at which the dialysis fluid parameter threshold (parameter extreme) has actually been reached in step d), or at which the venous pressure threshold PV or the arterial pressure threshold PA has been reached in step c) and the dialysis fluid parameter threshold has not yet been reached in step d), step d) being continued for a predetermined waiting time tx from the moment at which the venous pressure threshold PV or the arterial pressure threshold PA has been reached,
f) otherwise, returning to step b) preferably with a return unit.

Depending on the distance between the detection site and the dialyzer (seen in the direction of flow of the spent dialysis fluid) and on the flow velocity of the dialysis fluid, the waiting time may be zero, if the dead time Δt is virtually zero, or it may preferably be longer than/equal to the dead time Δt. If, in this case, one of the pressure thresholds PV, AV were reached during the blood flow alteration period t in the case of a current blood flow, only the time tx would additionally be allowed to elapse, so as to see whether a parameter extreme appears (subsequently) on the dialysis side. If this is the case, the respective actual blood flow (smaller than the current blood flow) would be determined. Otherwise, the current blood flow would be the optimum one.

Here, it should additionally be mentioned that the blood flow optimum value may also be slightly smaller than the current/actually measured blood flow for which one of the pressure thresholds PV, AV or the parameter extreme has been reached.

Furthermore, it should be pointed out that the waiting time tx need not necessarily correspond to the dead time Δt. In particular, the following holds true: waiting time tx≧dead time Δt. Preferably, the following holds true: waiting time tx=x·Δt (with x≧1).

It follows that, with the method according to aspects of the present invention, it can is be achieved that the blood cleansing machine is operated either
at the adjusted maximum blood flow (first criterion) or
approximately at the blood flow in the case of which the maximum admissible arterial pressure/venous pressure (second criterion) is reached or
approximately at the blood flow in the case of which a parameter extreme (third criterion) occurs/occurred
   (according to the criterion which is fulfilled first), even if the parameter extreme (third criterion) should occur only with a time delay relative to the other criteria and if a possibly already stored (preliminary) blood flow optimum value (Qb_optimum) (resulting from the first or the second criterion) is therefore reduced to the blood flow value at which the (only subsequently) ascertained parameter extreme (third criterion) occurred.

Preferred embodiments of the method according to aspects of the present invention comprise, as far as this is technically possible and reasonable, as a further feature or as a combination of further features that
step d) is carried out, continuously or in a clocked mode, in the course of the predetermined waiting time tx (theoretically assumed value which may correspond to the actual delay time or which at least approaches this delay time), when a threshold is reached in step c);
the blood flow alteration rate is adjusted in dependence on a predetermined/enterable blood flow start value Qb_start, the predetermined/enterable blood flow target value Qb_target, and possibly the predetermined blood flow alteration period t;
the predetermined blood flow start value Qb_start is 50 ml/min, and that preferably the predetermined blood flow target value Qb_target is 600 ml/min at the most;
the blood flow target value Qb_target is stored as a default value in a control device, inputted via a communication unit, read-in from a patient data card or to transmitted from a server;
the dead time Δt is predetermined in dependence on the blood flow alteration rate, the blood flow target value Qb_target, a dialysis fluid flow Qd, and parameters of the extracorporeal blood treatment machine/cleansing machine, preferably the dialysis machine (see preferably the parameter definition according to the description of the figures following hereinbelow);
the parameters of the extracorporeal blood treatment machine are inputted with the aid of the communication unit, read-in from a bar code or loaded from a server comprising data to be adjusted for the treatment of patients;
the dead time Δt and the blood flow target value Qb_target are stored as pairs of values in a value table in dependence on parameters of the extracorporeal blood treatment machine.

The corresponding extracorporeal blood treatment machine/cleansing machine, preferably dialysis machine, of the generic type has the following features preferably for carrying out the above described control method:
a dialyzer for blood cleansing,
at least one blood pump for creating an extracorporeal blood flow between a patient and the dialyzer,
at least one dialysis fluid pump for supplying the dialyzer with a dialysis fluid,
at least one venous blood pressure sensor downstream of (subsequent to) the dialyzer,
at least one arterial blood pressure sensor upstream of (prior to) the dialyzer,
at least one dialysis fluid sensor for detecting at least one dialysis fluid parameter (e.g. UV-absorption/absorbance) subsequent to (downstream of) the dialyzer at a certain flow distance from the dialyzer,
optionally, at least one blood flow sensor for detecting the extracorporeal blood flow, if it should not be possible to adjust said blood flow directly with the pump,
optionally, at least one dialysis fluid flow sensor for detecting a dialysis fluid flow, if it should not be possible to adjust said fluid flow directly with the pump.

According to aspects of the present invention, the blood treatment machine, e.g. the dialysis device, is further developed by
a communication unit for predetermining an extracorporeal blood flow target value Qb_target and, optionally, an extracorporeal blood flow start value Qb_start, and
a control/regulating unit for setting/adjusting an extracorporeal blood flow value of the blood flow (in dependence on the blood flow target value Qb_target), which comprises:
control means for altering (increasing/possibly decreasing) the extracorporeal blood flow Qb at a predetermined or selected blood flow alteration rate,
a comparator for comparing a (currently measured) venous pressure PV with a predetermined or selected venous pressure threshold, a (currently measured) arterial pressure PA with a predetermined or selected arterial pressure threshold, and the current blood flow Qb with the blood flow target value Qb_ta rget,
a detection unit for detecting at least one current dialysis fluid parameter/feature/characteristic (degree/amount of uremic toxins contained in a spent dialysis fluid, e.g. via UV-absorption/absorbance),
a determination unit for determining whether the detected current dialysis fluid parameter approaches/has reached a dialysis fluid parameter threshold (determination of the occurrence of a parameter extreme) and
an optimum blood flow value memory for storing as an optimum blood flow value Qb_optimum the blood flow in the case of which the dialysis fluid parameter threshold (parameter extreme) has been reached, or for temporarily storing as an optimum blood flow value Qb_optimum the blood flow in the case of which the venous pressure threshold or the arterial pressure threshold has been reached (and the dialysis fluid parameter threshold has not yet been reached), or for storing as an optimum blood flow value Qb_optimum the extracorporeal blood flow value Qb_target, if neither the parameter threshold nor the venous pressure threshold or the arterial pressure threshold has been reached (previously).
In addition, according to aspects of the present invention, the control/regulating unit is configured such that it will continue to operate at least the detection and determination unit over a waiting time tx even if the comparator recognized that the extracorporeal blood flow target value Qb_target or the selected venous/arterial pressure threshold has been reached at a specific moment in time, at which the waiting time tx is started, (and the blood flow increase has thus been stopped), so as to subsequently adjust/alter (re-reduce) the already stored preliminary optimum blood flow value Qb_optimum to the blood flow value at which a parameter extreme x, delayed by a delay time/dead time Δt, may possibly be ascertained. As regards the ratio between waiting time tx and dead time Δt, the above definitions apply.

Preferred embodiments of the blood treatment machine according to aspects of the present invention, in particular of a dialysis machine, comprise, as far as this is technically possible and reasonable, as a further feature or as a combination of further features that
the control device has a time extension unit for extending the determination executed by the determination unit in comparison with the comparing executed by the comparator by the predetermined or adjustable waiting time, tx, in particular if the comparator recognizes/has recognized that a threshold has been reached/exceeded;
the control device, preferably in the form of a blood pump control unit, calculates/selects the blood flow alteration rate in dependence on a predetermined blood flow start value Qb_start, the blood flow target value Qb_target, and a predetermined blood flow alteration period t;
the control device, preferably the blood pump control unit, sets the predetermined blood flow start value Qb_start to 50 ml/min and, optionally, the blood flow target value Qb_target to >50 to 600 ml/min at the most;
the control device, preferably the blood pump control unit, reads the blood flow target value Qb_target as a default value in a control unit, reads-in said blood flow target value Qb_target from a communication unit, from a patient data card or from a server;
the time extension unit determines the waiting time tx in dependence on the dead time Δt, which results from the blood flow alteration rate, the blood flow target value Qb_target, a dialysis fluid flow Qd and parameters of the blood treatment machine/dialysis machine;
the time extension unit reads-in the waiting time tx from a value table, said value table having stored therein, as pairs of values, the waiting time tx or the dead time Δt and the blood flow target value Qb_target in dependence on parameters of the blood treatment machine/dialysis machine.

The present invention has, inter alia, the following advantages:

The physician will be able to judge more precisely the blood flow to be selected for a treatment. A fast online method for ascertaining the blood flow is suggested for the first time. Making use of the method according to aspects of the present invention, application and control of the blood flow, e.g. for a dialysis treatment, can take place automatically. The blood flow is directly (online) adapted for maximizing the amount of toxins on the dialysis fluid side. To this end, the amount of toxins on the dialysis fluid side is monitored with an optical sensor (online). Likewise, the arterial and the venous pressure are monitored (online) with pressure sensors on the blood treatment machine/dialysis machine so as to guarantee the safety of the process.

Additional features and advantages of the present invention result from the description of preferred embodiments following hereinbelow, in which reference will be made to the enclosed drawing.

### BRIEF DESCRIPTION OF THE DRAWINGS

The invention is best understood from the following detailed description when read in connection with the accompanying drawings. Included in the drawings are the following figures:

FIG. 1 shows a schematically an embodiment of the blood treatment machine/dialysis machine according to aspects of the present invention.

FIG. 2 shows, for the purpose of illustration, the profile of the clearance against the blood flow, with and without disturbing effect.

FIG. 3 shows, for the purpose of illustration, the profile of the absorbance in the dialysis fluid against the blood flow, with and without disturbing effect.

FIG. 4 shows an embodiment of the (machine control) method according to aspects of the present invention, used for adjusting the current blood flow during dialysis.

FIGS. 5A and 5B each show, for the purpose of illustration, schematically the path of a possible disturbance in a dialysis system.

FIGS. 6A and 6B each show, for the purpose of illustration, schematically the determination of an extreme value in the intensity signal of a dialysis fluid sensor in a dialysis system.

### DETAILED DESCRIPTION OF THE PREFERRED EMBODIMENTS

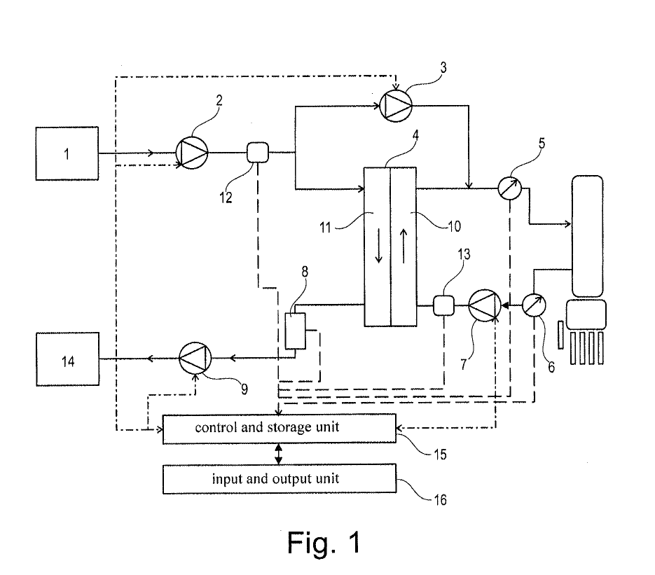

FIG. 1 shows a schematic flow chart of an extracorporeal blood treatment/blood cleansing/dialysis machine comprising a control device 15 in combination with a communication unit 16. A blood pump 7 extracts blood from the body of a patient via an access to the patient (shunt puncture site), which is schematically indicated as the patient's forearm on the right-hand side of FIG. 1. Optionally, the current blood pump rate may additionally be determined by a flow sensor 13 on the extracorporeal blood side. An arterial pressure sensor 6 monitors the current negative pressure on the suction side of the blood pump 7.

The blood pump 7 conveys the patient's blood through a dialyzer 4. In the course of this process, uremic toxins can pass from a blood side 10 to a dialysis fluid side 11 of the dialyzer 4 through a semipermeable membrane (not shown). The thus cleansed blood is then returned to the patient. A venous pressure sensor 5 monitors the venous pressure within the extracorporeal blood circuit downstream of the dialyzer 4.

Dialysis fluid pumps 2 and 9 generate the dialysis fluid flow through the dialyzer 4 on the dialysis fluid side 11 of the latter. The uremic toxins, which were transferred in the dialyzer 4 to the dialysis fluid side 11, are thus conducted past an optical sensor 8, which follows, preferably directly, the dialyzer 4 on the dialysis fluid side. The amount of uremic toxins transferred to the dialysis fluid side 11 can be measured with the optical sensor (UV sensor) 8. The intensity I and the absorbance A, respectively, in the spent dialysis fluid is here a measure for the current cleansing performance of the blood treatment machine/dialysis machine.

The spent dialysis fluid flows through the optical sensor 8 with a defined dialysis fluid flow Qd measured with a flow sensor 12 on the dialysis fluid side, said flow sensor 12 being disposed upstream of, preferably directly upstream of the dialyzer 4 in the present embodiment.

The hydraulic/fluidic structure of the above described blood treatment machine/dialysis machine corresponds largely to the prior art and is therefore fundamentally known. Also the operation of the blood treatment machine/dialysis machine is known per se from the prior art, so that a detailed description of the individual processes taking place at the dialyzer 4 is here not necessary.

The cleansing performance of the dialyzer 4 should normally be as high as possible. Since the extracorporeal blood flow represents, in addition to the dialysis fluid flow and the quality of the dialyzer 4, one of the control variables for optimizing the cleansing performance of the blood treatment machine/dialysis machine, the above described device and method according to aspects of the present invention serve to maximize the cleansing performance through adaptation of the extracorporeal blood flow.

To this end, the optical sensor 8 is used for analyzing the compensation process on the dialysis fluid side during and after the end of a defined blood flow alteration with the aid of the optical sensor 8. Hence, the measurement in question is, in principle, a measurement of transients, which is suitable for optimizing the blood flow in the case of hemodialysis (HD), hemodiafiltration (HDF), hemofiltration (HF) as well as “single needle cross over” (SNCO).

FIG. 1 shows which units are required in the machine for realizing the blood flow control according to aspects of the present invention and in which way these units communicate. An electronic communication unit 16 serves the user, on the one hand, to display and, on the other hand, to input treatment parameters (correspond to the parameters of the blood treatment machine), such as blood flow (start and/or target blood flow), dialysis fluid flow and/or pressure limits in the area where the blood enters and exits the patient. This is done e.g. via a graphical user interface of the machine, with a patient data card or via data transmission of the patient's individual treatment settings from an external patient data server.

These inputs are used by the control device 15 for controlling and/or regulating the pumps 2, 9, 7 and/or valves, which are not shown in detail. The information used for this purpose is partly information made available by the (blood) pressure sensors 5, 6, the flow sensor 12 on the dialysis fluid side, the flow sensor 13 on the blood side and the optical sensor 8. The control device 15 also serves the purpose of preprocessing the data provided by the sensors. Such preprocessing comprises e.g. the filtering and smoothing of the data (signals) and the extraction of parameters. Moreover, the control device 15 serves to store all the treatment parameters (machine parameters), and it also serves as a memory for all the information required for allowing the method for optimized blood flow adjustment to be applied. One example of such information are dialyzer-specific data, such as the blood-side volume (Vb) and the dialysis fluid-side volume (Vd) of the dialyzer 4, but also all the other characteristic curves and characteristic diagrams (e.g. dialyzer clearance, etc.) belong thereto.

The dialyzer 4 and the extracorporeal tube system/blood line system must be unequivocally identified for the respective case of use. This can, for example, be done with the aid of the communication unit 16, e.g. in that the user inputs the dialyzer model or in that a bar code provided on the dialyzer 4 is read-in. Alternatively, it would also be possible to produce a defined volume through automatic level setting in the dialyzer chambers.

Communication between all modules, such as the control device 15, the communication unit 16, the individual actors/pumps 2, 9, 7, etc., can take place in a unidirectional or bidirectional mode.

The intensity and the absorbance, respectively, of the spent dialysis fluid is a direct measure for the amount of uremic toxins transferred from the blood side to the dialysis fluid side. In this respect, the assumption that an increase in extracorporeal blood flow (Qb) will always lead to an increase in effective clearance (Ce) is taken as a basis. Hence, an increase in clearance would lead to a larger amount of uremic toxins on the dialysis fluid side and could be detected via a higher absorbance of the spent dialysis fluid. This assumption is, however, only applicable if no complications occur, through which the amount of uremic toxins will already be reduced at the patient's vascular access. Such a complication, e.g. local recirculation or shunt recirculation, has the effect that the amount of uremic toxins arriving at the dialysis fluid side will, in spite of an increase in blood flow, not increase or not increase to the same extent, but remain e.g. the same, increase to a lesser extent or even decrease. The relationship between clearance Ce and recirculation may here be described as follows:

Ce=(1−R)Cd/(1−R(1−Cd/Qb)) (1)

with Ce as effective clearance “from the patient's point of view” (does not necessarily correspond to the dialyzer clearance), R as recirculation and CD as clearance of the dialyzer. This means that, if recirculation/shunt recirculation occurs, the effective clearance will markedly lag behind the outright dialyzer clearance, which would be equal to the effective clearance for R=0.

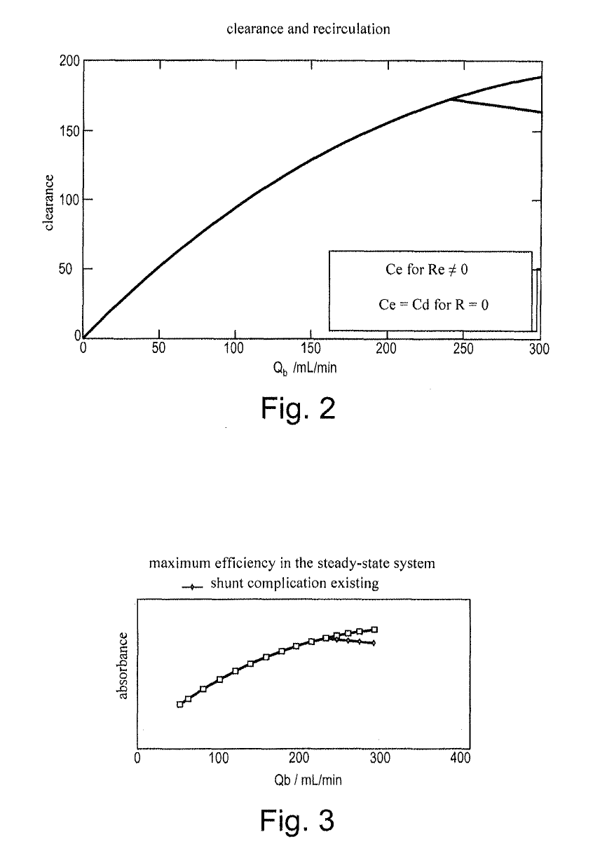

FIG. 2 shows, for the purpose of illustration, an example of reducing the effective clearance Ce of a dialyzer based on the assumption that a recirculation of 20% takes place in the case of a blood flow of 300 ml/min.

In FIG. 2 the clearance is shown as a function of the blood flow Qb according to equation (1). If the blood flow Qb increases, the effective clearance will increase as well. The clearance has a non-linear and, in the event that no recirculation occurs, a monotonically increasing profile. However, if recirculation occurs at the access to the patient, the recirculation effects will reduce the effective clearance Ce, this being shown in FIG. 2 as the line branching off/deviating downwards from the theoretical/ideal effective clearance in the case of high Qb values.

FIG. 3 shows, for the purpose of illustration, an example of a so-called steady-state absorbance, measured approx. 285 nm downstream of the dialyzer 4. The figure shows the dialysis-fluid-side absorbance against the blood flow. In the case of high Qb values, the reduction of the absorbance through recirculation effects is shown as a falling line.

The absorbance is equivalent to the amount of uremic toxins removed from the blood. The cleansing performance is therefore maximal, when the amount of toxins on the dialysis fluid side is maximal for defined Qb, Qd. Hence, the cleansing performance will also be maximal, when the absorbance and/or the intensity at the measuring channel of the sensor is/are minimal.

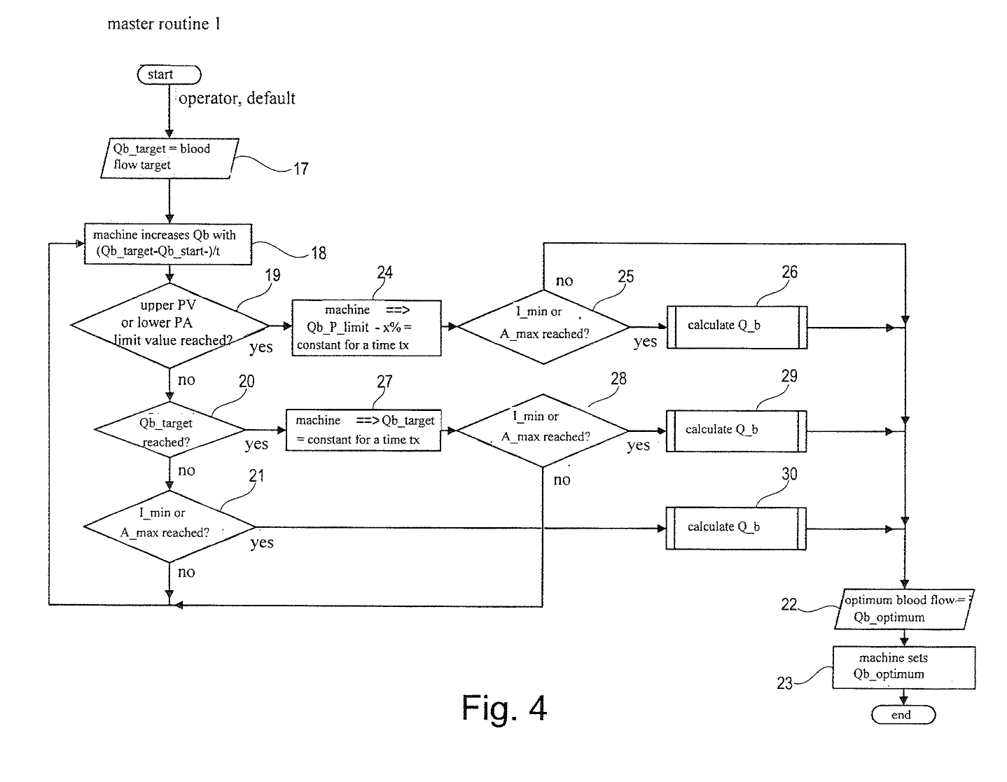

FIG. 4 shows an embodiment of the method according to aspects of the present invention. In the case of the method according to aspects of the present invention, the blood flow which is most advantageous for the treatment is obtained by combining a, preferably linear, alteration of the blood flow, the analysis of the pressure signals and the optical measurement.

Starting from a selected or preset blood flow start value Qb_start, the embodiment of the (machine control) method according to FIG. 4 comprises the steps of
17 predetermining a blood flow target value Qb_target,
18 altering the blood flow Qb with a predetermined, possibly linear blood flow alteration rate, preferably through a blood pump control unit or a flow regulating valve or the like,
19 comparing a (blood side) venous pressure PV with a selected or predetermined venous pressure threshold and a (blood side) arterial pressure PA with a selected or predetermined arterial pressure threshold,
20 comparing the current blood flow Qb with the blood flow target value Qb_target, preferably with a comparator unit,
21 detecting at least one dialysis fluid parameter (e.g. absorbance) and preferably tracking the parameter profile by a detection unit and determining a possible occurrence of a parameter extreme through a determination unit,
22 storing the optimum blood flow value Qb_optimum based on the results obtained in steps 19 to 21,
23 operating the machine with the optimum blood flow value Qb_optimum for the time being,
24 operating the machine with the blood flow value Qb_P_limit−x % during a predetermined waiting time tx,
25 subsequent/continuous determination of a possible occurrence of a parameter extreme by the determination unit at least during the waiting time tx or longer than that,
26 recalculating/readjusting the blood flow value Q_b (in the event that a parameter extreme was subsequently determined),
27 operating the machine with the blood flow target value Qb_target during a predetermined waiting time tx,
28 subsequent/continuous determination of a possible occurrence of a parameter extreme by the determination unit at least during the waiting time tx or longer than that,
29 recalculating/readjusting the blood flow value Q_b (in the event that a parameter extreme was subsequently determined) and
30 recalculating/readjusting the blood flow value Q_b (in the event that a parameter extreme was determined).

The above steps will be explained in detail hereinbelow.

In step 17, a target value for the blood flow, Qb_target, is inputted (e.g. >50-600 ml/min). The blood flow target value, Qb_target, may be stored in the control device 15 as a default value, inputted via the communication unit 16, read-in from a patient data card (not shown) or transmitted from a server (not shown).

In step 18, the current blood flow Qb is increased (continuously or step by step at a specific rate of increase) from a predetermined value Qb_start (e.g. 50 ml/min) to the value Qb_target within a predetermined period t. The predetermined value Qb_start is e.g. 50 ml/min and is a fixed value, whereas the value Qb_target (e.g. 300 ml/min) may, as mentioned above, be predetermined by the user with the communication unit 16 or transmitted from a patient data card or a server.

With the control device 15 parameters are retrieved, continuously or in a clocked mode, said parameters being used as criteria for terminating or controlling the blood flow increase. These criteria are the following ones:

i. one of the pressure value limits (upper limit value venous pressure PV and/or lower limit value arterial pressure PA in the extracorporeal blood circuit) is reached/exceeded,

ii. an intensity minimum and/or absorbance maximum occurs in the signal of the optical sensor,

iii. the demanded high blood flow level Qb_target is reached/exceeded.

Hence, in step 19 it is queried whether an upper limit value for the venous pressure, PV, and/or a lower limit value for the arterial pressure, PA, has been reached/exceeded.

In step 20, it is additionally queried whether the target value of blood flow, Qb_target, has been reached. As mentioned above, the target value of blood flow, Qb_target, may be adjusted by the user manually via the communication unit 16 or it may be predetermined as a default value, said default value being loaded from the control device 15.

Likewise, it is queried in step 21 whether an intensity minimum I_min and/or an absorbance maximum A-max has been identified in the signal of the optical sensor 8 for the concentration of uremic toxins in the dialysis fluid downstream of the dialyzer 4.

FIG. 4 shows the additional steps of the method depending on which of the queries 19 to 21 first leads to branching in the sequence.

If the query in step 19 is “yes”, i.e. if it is recognized that one of the two limit pressures PV and PA has been reached, a blood flow, in the case of which the pressure will remain within the pressure limits, will be adjusted (first temporarily). This blood flow is then kept constant for a waiting time tx. This waiting time tx approximately corresponds to a delay or dead time to be expected, which elapses until a parameter extreme occurs at the dialyzer 4 via the dialysis drain line from the dialyzer 4 to the (absorption) sensor 8. Irrespectively of this, the signal of the optical sensor 8 is, optionally, still permanently evaluated until the compensating process on the dialysis fluid side has been fully terminated, i.e. until a stable final level has been established and the signal at the optical sensor 8 no longer changes.

If the control device 15 does not identify an extreme value in the form of an intensity minimum or an absorbance maximum, the blood flow adjusted (first provisionally) as a constant blood flow will also be the optimum blood flow for the treatment. This is queried in step 25, after which the method continues, in the case of a negative result, directly with step 22. In step 22, the optimum blood flow is stored as optimum blood flow value Qb_optimum in a blood flow optimum value memory, and the machine is adjusted “permanently” to this value in step 23. The method has thus been finished and is terminated.

If, however, an extreme value in the form of an intensity minimum and/or an absorbance maximum is recognized by the control device 15 in the signal of the optical sensor in step 25, the optimum blood flow will be calculated on this basis. In so doing, the blood flow is calculated (reconstructed) by the control device 15 with the aid of the moment in time at which the extreme occurs or has occurred (time of occurrence). This is done in step 26, in which the blood flow is then provided as a new blood flow value Q_{—b}.

If it turns out in step 21 that an intensity maximum or minimum I_min and/or an absorbance minimum or maximum A_max has been reached, the control device 15 will detect (with a certain delay), e.g. during evaluation of the transient signal from the optical sensor 8, the occurrence of an extreme value. On this basis, the control device 15 will then calculate in step 30 as well as in steps 22 and 23 the new blood flow value Q_b and, based on this value, the optimum blood flow, optimum blood flow value Qb_optimum.

If it turns out in step 20 that the blood flow target value Qb_target for the blood flow has been reached (for the time being, without an extreme value having been ascertained), the blood flow is kept constant (provisionally) for a waiting time tx in step 27. The evaluation of the signal of the optical sensor 8 through the control device 15 is nevertheless continued.

If, subsequently, i.e. after the provisional adjustment of the blood flow to the blood flow target value Qb_target, a parameter extreme should nevertheless be ascertained (with a certain delay) preferably within the waiting time tx, a new blood flow value Q_{—b} (corresponds approximately to the blood flow that prevailed when the extreme actually occurred) will be calculated in this case in step 29, and, depending on this new blood flow value Q_{—b}, the optimum blood flow value Qb_optimum will, in turn, be determined in step 22 and the machine will, for the time being, be operated with this value in step 23, so that the method comes here to an end. If the control device 15 was not able to find/ascertain an extreme value in the form of an intensity minimum and/or absorbance maximum preferably within the waiting time tx or within the waiting time tx and beyond, a return to step 18 will take place in step 28, and the blood flow will be increased still further, until either the venous threshold PV or the arterial threshold PA is reached in step 19 or the dialysis fluid threshold I_min and/or A_max is reached in step 21. Alternatively, also the physician in charge or the attending operator may be requested to specify a new blood flow target value Qb_target.

The monitoring of the pressure values PV, PA serves here the purpose of preventing said pressure values from exceeding or falling below the admissible lower arterial and upper venous pressure, i.e. it serves the safety of the patient. Both pressure values are influenced by the interaction of needles, the puncturing situation and the patient's vascular status. The monitoring of the pressure values is executed with the control device 15.

The optical sensor 8 on the dialysis fluid side measures the intensity and the absorbance of the spent dialysis fluid in dependence on the washed-out uremic toxins dissolved therein. The control device 15 searches for an extreme point in the sensor signal, which extreme point would only occur in response to complications, e.g. a local recirculation, such as a shunt recirculation.

The signals of the pressure sensors 5, 6 and of the optical sensor 8 are the basis for the actions triggered by the control device 15. All sensor data can be processed with the control device 15, e.g. smoothed or filtered by a lowpass filter. FIG. 4 shows especially for the query 20 “Qb_target reached?” and the query 19 “PV or PA reached?” that conclusions with respect to the optimum blood flow cannot be drawn immediately afterwards. The reason for this is the following:

the values for judging the query 20 “Qb_target reached?” and the query 19 “PV or PA reached?” are immediately available. This, however, does not equally apply to the data of the optical sensor 8.

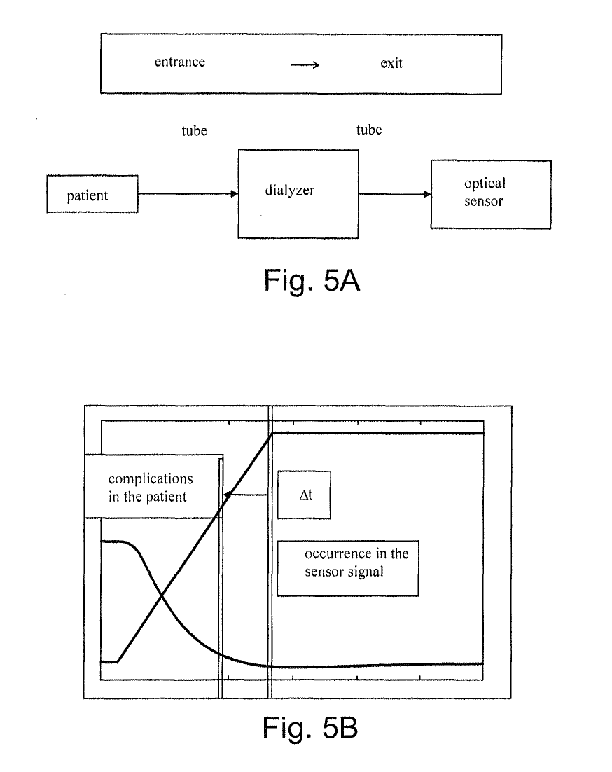

The temporal sequence of a pressure change and of the reaction at the optical sensor 8 will be explained in the following making reference to FIG. 5A and 58. FIG. 5A schematically indicates the direction in which the processes will be explained in the following. The patient is at the entrance to the system. He is connected to the dialyzer 4 via a blood-side tube system. The dialyzer 4, in turn, is connected via a further dialysis-side tube system to the optical sensor 8, which monitors predetermined parameters (absorbance, absorption, etc.) of the exit-side, spent dialysis fluid. If a change occurs at the patient, e.g. a local recirculation, such as shunt recirculation with reduction of the effective clearance, this change must first make itself felt over the path to the optical sensor. This means that conveyance through the arterial tube section, the dialyzer and through the dialysis-fluid-side tubing must take place before the effect of this change at the entrance to the system (access to the patient) can be detected by the optical sensor 8. This delay in time is shown in FIG. 5B.

In FIG. 5B the signal profile is plotted against time. From FIG. 5B it can be seen that the effect of a local recirculation at the access to the patient, such as a shunt recirculation, can only be measured by the optical sensor 8 with a considerable delay in time. This is the reason for the fact that a waiting time is provided after the query 20 “Qb_target reached?” or the query 19 “PV or PA reached?” and prior to the queries 27 or 28 “I_min and/or A_max reached?”, so as to avoid that an incorrect blood flow will be adjusted.

As can be seen from FIGS. 5A and 5B, this means that, for system-inherent reasons, a change occurring at the access to the patient/the patient's shunt, cannot be detected by the optical sensor 8 until it has travelled through the tube system on the blood side, the dialyzer 4 and the dialysis-fluid-side path to the sensor 8. Hence, an event occurring at the moment in time t will only be detected with delay (dead time) at the moment in time t+Δt.

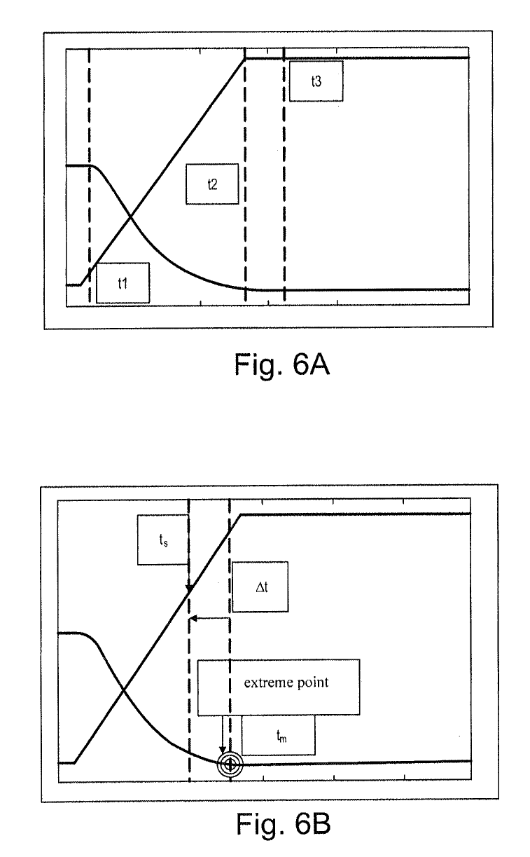

In FIG. 6A and FIG. 6B, blood flow determination is explained exemplarily. In both FIGS. 6A and 6B, the increase in blood flow (rising line in FIGS. 6A and 6B) as well as the intensity signal of the optical sensor 8 (decreasing curve in FIGS. 6A and 6B) are plotted against time. In FIG. 6A the blood flow is increased from the moment in time t1 onwards. The moment in time t2 identifies the end of blood flow increase, whereas at t3 the end of the change in intensity at the optical sensor 8 is reached. Since the optical sensor 8 is arranged on the dialysis fluid side, a delay in time will always occur. Blood-side changes in the system, which influence the amount of toxins in the dialysis fluid, cannot be measured at the optical sensor 8 immediately after having occurred. Conveyance through the blood-side tube system and the dialyzer 4, etc. must here additionally be taken into account. In addition, compensation processes take place, which depend on the volumes (blood side, dialysis fluid side) in the dialyzer 4.

In FIG. 6B, the delay time in the signal is shown. Through the increase in blood flow a change in concentration at the access to the patient was here induced at the moment in time ts. The reason for this change in concentration may e.g. be a local recirculation/shunt recirculation. This has the effect that less substances will be able to arrive at the dialysis fluid side. If this change in concentration occurs at the moment in time ts during the increase in blood flow, the detected intensity will first start to decrease and will then re-increase after some time, although the blood flow is still increased (in this context, it should be mentioned that, in response to an increase in blood flow, the intensity should, as a matter of principle, always decrease, only the subsequent increase would indicate the inducing of recirculation). In FIG. 6B, this change in concentration manifests itself in the formation of an extreme point (at the moment in time tm) in the sensor signal of the optical sensor 8. The above described delay time between occurrence on the entrance side and measurement of the effect on the exit side is designated as Δt in FIG. 6B (and may possibly correspond to the waiting time tx; ideally the following holds true: Δt≦tx).

The occurrence time tm of this extreme point is related to the time is at which the complication occurs at the entrance. If there is a change at the entrance, the following holds true:

tm>ts

ts=tm−Δt.

When Δt is known, the optimum treatment blood flow can be ascertained from the knowledge of tm, since this is the blood flow Qb (tm−Δt).

This dead time Δt depends on the selected blood flow alteration Qb(t) and Qb_target, respectively, the dialysis fluid flow Qd and the volumes involved, e.g. in the dialyzer 4 (Vbeff effective blood-side volume, Vdeff effective dialysis-fluid-side volume) and in the tube system.

Hence, the following holds true:

Δt=f(Qb(t), Qd, Vbeff, Vdeff, Vtube_arterial).

Qb(t), Qd, Vbeff, Vdeff, as well as Vtube_arterial are known quantities, which have been taken e.g. from laboratory measurements or data sheets. To this end, the dialyzer 4 and the blood-side tube system are identified prior to the treatment so that the relevant tables can be accessed. The identification of the dialyzer 4 and of the tube system is carried out via the communication unit 16 through the user, the reading in of a bar code or the loading of data from a server, etc.

If it should not be desired to calculate Δt from the quantities Qb, Qd, Vtube_arterial, Vbeff and Vdeff, Δt is directly stored in a table, since the dependence of Δt and Qb_target is known from laboratory measurements, when the dialyzer 4 and the tube system have simultaneously been identified (e.g. through user input). Qd must be known as well. The dialysis fluid flow Qd is metrologically detected at all times. Table 1 shows an example for the assignment of the values of Δt to various Qb_target in the case of a dialysis fluid flow of 500 ml/min. In this example the dialyzer 4 and the tube system as well as the dialysis fluid flow Qd are already known. Hence, a lookup table is used, which comprises the values of Δt for the respective configuration of the dialyzer 4 and of the tube system as well as Qd.

TABLE 1

| Qb_target [ml/min] | Δt [s] |
|---|---|
| 300 | 20 |
| 400 | 25 |

The present invention provides for the first time a device in which the blood flow is adjusted automatically on the basis of the effective clearance and which additionally guarantees, through pressure monitoring, that there will be no risk for the patient. to This means, in more detail, that the situation at the access to the patient, e.g. during application and the adjustment of the blood flow, can be monitored directly (online). This means equally that any blood-flow-induced decrease in the effective clearance can be detected (virtually) instantaneously and that the blood flow can thus be adjusted to the maximum therapeutic effect. In other words, an optimization of the therapy to the maximum clearance can be accomplished with the method according to aspects of the present invention. Furthermore, due to the analysis of the compensation process, which is measured by an optical sensor, (transient measurement) on the dialysis fluid side, regulating for optimum blood flow is executed. Even if complications should occur at the access, the optimum dialysis treatment of each patent can be guaranteed. Through regulating/controlling the blood flow such that the maximum cleansing performance is accomplished and through the resultant possibility of reducing the dialysis fluid flow until the desired Kt/V has been reached (specified through Kt/V prediction), dialysis fluid can be saved. This applies especially to cases where the Kt/V is higher than the desired quality level. Furthermore, the above described process can also be re-initiated at any time in the course of the treatment, if this should be desired by the attending staff (To this end, the change in blood flow can be effected in both directions, namely, increase/decrease). By storing the ascertained blood flows and by trend evaluation, the shunt situation can be monitored. A recirculation will be detected automatically through determination of the extreme point. The measurement times can be kept short, so that the measurement period will be less than 4 minutes. Due to the already existing sensor system, the costs to be expected can be kept low.

Since measurement takes place on the dialysis fluid side, no additional effort on the part of the nursing staff is required for preparing the dialysis and for placing the blood tube system into the sensors, and in addition the nursing staff's workload is reduced by an automatic application procedure.

In the case of a preferred embodiment (not shown), a red detector for detecting blood is used when the device is applied to the patient. A blood pump rate of 50 ml/min up to the access to the patient can be achieved. An increase in blood flow up to a prescribed blood flow value can be initiated automatically or manually by the operating staff (in FIGS. 6A and 6B to Qb_target 600 ml/min). The optical sensor monitors the intensity in the outgoing dialysis fluid, and the control device 15 evaluates the signal continuously and ascertains e.g. the slope. Pressure sensors monitor the admissible pressure limits. The control device 15 evaluates the data and determines the time at which an extreme occurs or it ascertains the occurrence of some other termination criterion (e.g. PV or PA reached). With the aid of a lookup table, such as table 1, the value of is is determined from tm−Δt. For selecting the correct lookup table, the configuration of the dialyzer and of the tube system as well as the Qd must be known.

The present invention consists of a device and of the related method for determining the optimum blood flow, preferably at the beginning of the therapy. This measurement can be carried out without any additional equipment being required, since the dialysis machine is already provided with all the necessary actuators and sensors. The blood flow, in the case of which a maximum of uremic substances will be transferred from the blood side to the dialysis fluid side, is determined by the reaching of one of three criteria according to the above description, when the blood flow is altered. One criterion is the reaching of the pressure limits PV and PA, the other criterion is the detection of the extreme values I_min and A_max and the last criterion is, finally, the reaching of the pre-adjusted maximum blood flow (target blood flow). Since, making use of the method according to aspects of the present invention, the blood flow can essentially be maintained at its maximum value, the dialysis fluid flow can be reduced for achieving the same cleansing performance, so that this may possibly open up a savings potential (usually in cases where it turns out during the treatment that the demanded dialysis dose is exceeded).

The blood treatment machine/dialysis machine according to aspects of the present invention thus comprises a dialyzer 4 for blood cleansing. For maintaining an external blood circuit, at least one blood pump 7 is provided, which creates a blood flow between a patient and the dialyzer 4. On the other side of the dialyzer 4, at least one dialysis fluid pump 2, 9 is provided, with which the dialyzer 4 is supplied with a dialysis fluid. For monitoring the dialysis process, at least one venous blood pressure sensor 5 is provided subsequent to (downstream of) the dialyzer 4. Analogously, the blood treatment machine/dialysis machine preferably comprises at least one arterial blood pressure sensor 6 prior to (upstream of) the dialyzer 4 and preferably at least one dialysis fluid sensor (optical sensor) 8 for detecting at least one dialysate parameter subsequent to (downstream of) the dialyzer 4. On the blood side of the dialyzer 4, preferably at least one blood flow sensor 13 is used for detecting a blood flow. Furthermore, the blood treatment machine/dialysis machine preferably comprises at least one dialysis fluid flow sensor 12 for detecting a dialysis fluid flow.

Via a communication unit 16, a blood flow target value, Qb_target, is predetermined. A control device 15 serves to adjust an optimum blood flow value in dependence on the pre-adjusted blood flow target value, Qb_target, the detected upper and lower blood pressures PA, PV as well as the possible detection of a parameter extreme. According to aspects of the present invention, the control device 15 comprises a blood pump control unit (not shown) for altering the blood flow, Qb, at a predetermined blood flow alteration rate or a flow regulating valve. This rate is especially (Qb-target−Qb_start)/t. A comparator unit (not shown) (inside the control device 15) is used for comparing a venous pressure PV with a venous pressure threshold, an arterial pressure PA with an arterial pressure threshold and the current blood flow Qb with the blood flow target value Qb_target. A determination unit (not shown) (constituent part of the control device 15) is provided for determining a parameter extreme in the spent dialysis fluid from the detected parameter values provided by the sensor 8. An optimum blood flow value memory (not shown) (again a constituent part of the control device 15) serves to store an optimum blood flow value Qb_optimum, e.g. in dependence on the blood flow which actually prevailed at the time of occurrence of the parameter extreme and preferably in dependence on the data listed in the lookup table and relating to Qb_target and Δt (actual delay time), if the determination unit recognized, possibly within the waiting/extension time tx, that the parameter extreme (dialysis fluid parameter threshold) was reached or if the comparator unit recognized that the venous pressure threshold or the arterial pressure threshold was reached and the determination unit recognized, possibly again within the waiting/extension time tx, that the parameter extreme (dialysis fluid parameter threshold) was not reached. If none of the above conditions is fulfilled, a return unit (not shown) will continue to alter (increase) the blood flow, Qb, at the same predetermined blood flow alteration rate as before, until the adjusted blood flow Qb_target, at the most, has been reached.

Preferably, a delay/extension unit (not shown) (constituent part of the control device 15) is provided between the comparator unit and the determination means/unit for delaying/extending the determination process executed through the determination unit by the (predetermined) waiting/extension time tx, if, as has already been described hereinbefore, the comparator unit has recognized, provisionally, that a pressure threshold has been reached.

In other words, when a pressure threshold has been reached, the determination unit will continue the determination process for the extension time tx so as to delay a decision on the existence of a parameter extreme to the end of the extension time tx. If it should then turn out that a parameter extreme did not exist in the spent dialysis fluid, the initially stored blood flow, at which the pressure threshold has been reached, will be maintained. If, however, the existence of a parameter extreme should subsequently be detected with delay, the blood flow at the real moment in time at which the parameter extreme occurred can be determined on the basis of the lookup table stored in advance, the blood flow optimum value being then defined in accordance with this blood flow.

Preferably, the blood pump control unit calculates the blood flow alteration rate in dependence on a predetermined blood flow start value Qb_start, the blood flow target value Qb_target and a predetermined blood flow alteration period, t. The blood flow target value Qb_target can read out as a default value, read in by a communication unit, read in from a patient data card or read in from a server.

The delay/extension unit determines preferably the waiting/extension time tx in dependence on the blood flow alteration rate, the blood flow target value Qb_target, a dialysis fluid flow Qd and parameters of the blood treatment machine/dialysis machine. In particular, the delay unit can read-in the waiting/extension time tx from the data/value table (lookup table), the delay time Δt and the blood flow target value Qb_target being stored in the data/value table as pairs of values in dependence on parameters of the blood treatment machine/dialysis machine so that the condition tx≧remains satisfied.

1-15. (canceled)

16. A machine control method for adjusting a blood flow in an extracorporeal blood treatment machine, said method comprising the steps of:
a) predetermining a blood flow target value (Qb_target);
b) altering the blood flow (Qb) at a predetermined or selected blood flow alteration rate;
c) comparing a venous pressure (PV) with a venous pressure threshold, an arterial pressure (PA) with an arterial pressure threshold, and the current blood flow (Qb) with the blood flow target value (Qb_target);
d) determining if a dialysis fluid parameter has reached a dialysis fluid parameter extreme;
e) storing the current blood flow (Qb) as an optimum blood flow value (Qb_optimum) in an optimum blood flow value memory, if the dialysis fluid parameter extreme has been determined, or if the venous pressure threshold or the arterial pressure threshold has been reached and the dialysis fluid parameter extreme has not been determined, and
f) storing the blood flow target value (Qb_target) as the optimum blood flow value (Qb_optimum), if neither the pressure thresholds nor the dialysis fluid parameter extreme has been reached.

17. The method according to claim 16, wherein, after a predetermined waiting time (tx), step d) is executed after step c), if a threshold is reached in step c), or wherein step d) is executed, in a clocked mode or continuously, beyond a predetermined extension time (tx) after step c), if a threshold or the blood flow target value (Qb_target) is reached in step c).

18. The method according to claim 17, wherein the optimum blood flow value (Qb_optimum) is determined in dependence on a new blood flow value (Q_b) or in dependence on the predetermined blood flow target value (Qb_target) in accordance with a data table stored in advance, and stored in the optimum blood flow value memory, if a dialysis fluid parameter extreme has been reached after the waiting time or during/after the extension time (tx), the new blood flow value (Q_b) corresponding to the blood flow that actually prevailed at the time of reaching the dialysis fluid parameter extreme.

19. The method according to claim 16, wherein the blood flow alteration rate depends on a predetermined blood flow start value (Qb_start), the blood flow target value (Qb_target) and a predetermined blood flow alteration period (t).

20. The method according to claim 19, wherein the blood flow alteration rate is adapted to be manually inputted.

21. The method according to claim 19, wherein the predetermined blood flow start value (Qb_start) is 50 ml/min.

22. The method according to claim 16, wherein the blood flow target value (Qb_target) is stored in a control device as a default value, inputted via a communication unit, read-in from a patient data card or transmitted from a server.

23. The method according to claim 16, wherein the waiting/extension period (tx) is predetermined or adjustable in dependence on a delay time (Δt) between reaching the dialysis fluid parameter extreme and its detection at a detection site.

24. The method according to claim 23, wherein the waiting/extension period is dependent on the blood flow alteration rate, the blood flow target value (Qb_target), a dialysis fluid flow (Qd) and parameters of the blood treatment machine.

25. The method according to claim 23, wherein the parameters of the blood treatment machine are inputted via the communication unit, read-in from a bar code, or loaded from a server comprising data to be adjusted for the treatment of patients.

26. The method according to claim 23, wherein the delay time (Δt) and the blood flow target value (Qb_target) are stored as pairs of values in a lookup table.

27. An extracorporeal blood treatment machine operated according to a control method according to claim 16 and which comprises:
a dialyzer,
at least one blood pump for creating an extracorporeal blood flow between a patient and the dialyzer,
at least one dialysis fluid pump for supplying the dialyzer with a dialysis fluid,
at least one venous blood pressure sensor downstream of the dialyzer,
at least one arterial blood pressure sensor upstream of the dialyzer,
at least one dialysis fluid sensor for detecting at least one dialysis fluid parameter downstream of the dialyzer; and
a control device for adjusting the blood flow value of the blood flow in dependence on the blood flow target value (Qb_target),
wherein the control device comprises:
a control/regulating unit for altering the current blood flow (Qb) at a predetermined or selected blood flow alteration rate,
a comparator unit for comparing the venous pressure (PV) with the venous pressure threshold, the arterial pressure (PA) with the arterial pressure threshold, and the current blood flow (Qb) with the blood flow target value (Qb_target),
a determination unit for determining if a dialysis fluid parameter extreme has been reached from a number of measurement values through the dialysis fluid sensor, an optimum blood flow value memory for storing an optimum blood flow value (Qb_optimum), if a dialysis fluid parameter extreme has been reached, or if the comparator unit recognizes that the venous pressure threshold, the arterial pressure threshold or the blood flow target value has been reached and the determination unit has not, or not yet determined that the dialysis fluid parameter extreme has been reached.

28. The blood treatment machine according to claim 27, wherein the control device additionally comprises:
a delay/extension unit for delaying/extending the determination process of the determination unit by at least a predetermined waiting/extension time (tx), if the comparator unit recognized that a threshold has been reached and if an associated blood flow has been temporarily stored as the optimum blood flow value (Qb_optimum) by the optimum blood flow value memory.

29. The blood treatment machine according to claim 27, wherein the optimum blood flow value (Qb_optimum) is determined in dependence on a new blood flow value (Q_b) or in dependence on the blood flow target value (Qb_target) according to a previously stored data table and is stored in the optimum blood flow value memory, if the determination unit determines the dialysis fluid parameter extreme has been reached during or after the waiting/delay time (tx), the new blood flow value (Q_b) corresponding to the blood flow that actually prevailed at the time of reaching the parameter extreme.

30. The blood treatment machine according to claim 27, wherein the control device calculates the blood flow alteration rate in dependence on a predetermined blood flow start value (Qb_start), the blood flow target value (Qb_target) and a predetermined blood flow alteration period (t).

31. The blood treatment machine according to claim 27, wherein the control device sets the predetermined blood flow start value (Qb_start) to 50 ml/min, the blood flow target value (Qb_target) being stored as a default value in the control device, or read-in by a communication unit, or read-in from a patient data card or from a server.

32. The blood treatment machine according to claim 27, wherein the delay/extension unit determines the waiting/extension time (tx) in dependence on the blood flow alteration rate, the blood flow target value (Qb_target), a dialysis fluid flow (Qd) and parameters of the blood treatment machine.

33. The blood treatment machine according to claim 27, wherein the delay/extension unit reads-in the waiting/extension time (tx) from a previously stored data or value table in dependence on a delay time (Δt) between the actual occurrence of the dialysis fluid parameter extreme and its detection at a detection site, the delay time (Δt) and the blood flow target value (Qb_target) being stored in said value table as pairs of values in dependence on parameters of the blood treatment machine.
